(19) United States
(12) Patent Application Publication
(10) Pub. No.: US 2010/0229251 A1
(43) Pub. Date: Sep. 9, 2010

(54) Methods and Compositions for Detecting and Treating End-Stage Cardiomyopathy Using Claudin-5

(75) Inventors: Jill A. Fortney, Westerville, OH (US); Philip F. Binkley, Columbus, OH (US); Paul M. Janssen, Dublin, OH (US); Tessily A. Mays, Pataskala, OH (US); Jamie L. Sanford, Worthington, OH (US)

Correspondence Address: MACMILLAN SOBANSKI & TODD, LLC, ONE MARITIME PLAZA FIFTH FLOOR, 720 WATER STREET, TOLEDO, OH, 43604-1619, US

(73) Assignee: THE OHIO STATE UNIVERSITY RESEARCH FOUNDATION, Columbus, OH (US)

(21) Appl. No.: 12/681,196

(22) PCT Filed: Oct. 2, 2008
(86) PCT No.: PCT/US08/78550

§ 371 Date: May 10, 2010

(30) Foreign Application Priority Data
Oct. 3, 2007 (US) 60997456

(51) Int. Cl. A01K 67/00; G01N 33/68; A61K 31/00; C12Q 1/02; G01N 33/53; A61P 9/00

(52) U.S. Cl. 800/9; 436/86; 514/789; 435/29; 435/7.1

(57) ABSTRACT

The present invention provides a method for diagnosis end-stage cardiomyopathy includes measuring expression of claudin-5 levels in a patient suspected of suffering from end-stage cardiomyopathy as well as a method for treating end-stage cardiomyopathy includes administering an effective amount of a composition effectively upregulates the claudin-5 or inhibits degradation of claudin-5

### CROSS-REFERENCE TO RELATED APPLICATIONS

This application claims the benefit of U.S. Provisional Application No. 60/997,456, filed Oct. 3, 2007, the disclosure of which is incorporated herein by reference.

### STATEMENT REGARDING FEDERALLY SPONSORED RESEARCH

This invention was made with no Government support and the Government has no rights in this invention.

### TECHNICAL FIELD AND INDUSTRIAL APPLICABILITY OF THE INVENTION

This invention is directed to certain novel compounds, methods for producing them and methods for detecting end-stage cardiomyopathy. More particularly, this invention is directed to developing compounds useful to upregulate claudin-5 or inhibit its proteolysis to treat patients with heart failure and patients with cardiac dysfunction prior to progression to heart failure.

### BACKGROUND OF THE INVENTION

Over 500,000 cases of heart failure are diagnosed annually in the U.S. and there are currently over 5 million patients in the U.S. in heart failure. Current pharmacological treatments are not successful in all patients and the lives of those patients not helped by current available drugs can only be saved by successful heart transplants. Heart failure currently contributes to 300,000 deaths per year.

Dilated and ischemic cardiomyopathies (DCM and ICM) can be caused by a multitude of different primary factors, all of which result in end stage cardiac failure. Thirty to forty percent of cardiomyopathies are familial and have been found to result from genetic mutations in cytoskeletal, sarcomeric, or other classes of proteins¹. The causes of the remaining cardiomyopathies are still unidentified and the pathways leading from cardiomyopathy to end stage failure are unknown.

Recent evidence from rodent studies suggests an important mechanistic connection between cell junction protein remodeling and cardiomyopathy. Cardiomyocytes are connected end-to-end at intercalated discs, which contain three types of cell junctions. Adherens junctions and desmosomes mechanically attach cardiomyocytes, while gap junctions ionically couple cardiomyocytes. Muscle LIM protein (MLP) knockout mice and tropomodulin-overexpressing transgenic mice both show dilated cardiomyopathy (DCM) with a concomitant upregulation of all adherens junctions proteins and a minor reduction in gap junction proteins². Transgenic mice overexpressing the adherens junction protein N-cadherin, or the non-heart isoform, E-cadherin, in heart develop DCM³. The gap junction protein connexin-43 (Cx43) is reduced in the hearts of these cadherin transgenic mice and correlates with the severity of cardiomyopathy. Mice with a conditional knockout of the N-cadherin gene in adult heart show an absence of adherens junctions and desmosomes and alterations of connexins⁴. These mice have mild DCM and impaired cardiac function and die of sudden cardiac death several weeks after the loss of N-cadherin protein.

An increasing amount of human data also supports a role for cell junction proteins in DCM and heart disease.^{5, 6} Mutations in the adherens junction-associated protein metavinculin cause DCM and hypertrophic cardiomyopathy, and there is a lack of expression of this isoform in other DCM hearts^{6, 7}. Expression of vinculin, another adherens junction-associated protein, is upregulated in some DCM patients⁸. Cx43 displays altered localization in human congestive heart failure and is downregulated in dilated, hypertrophied and ischemic cardiomyopathic hearts⁹⁻¹¹. Mutations in gamma-catenin, desmoplakin and plakophilin 2 lead to arrhythmogenic right ventricular cardiomyopathy [12; and reviewed in 13]. Other cell junction proteins remain uncharacterized in heart disease.

The level of the cell junction protein claudin-5 is greatly reduced in hearts from utrophin/dystrophin-deficient cardiomyopathic mice¹⁴⁻¹⁶. Cardiac contractile dysfunction in these mice mimics the phenotype of end-stage heart failure¹⁷. Claudin-5 is known to be a structural component of endothelial tight junctions, which are cell-cell junction structures absent from cardiomyocytes. Claudin-5 is present, however, at the lateral membranes of cardiomyocytes at their junction with the extracellular matrix and also in the endothelial layer of cardiovasculature. Claudin-5 is decreased at the lateral membranes of cardiomyocytes, but not from cardiovasculature in hearts from utrophin/dystrophin-deficient cardiomyopathic mice. This loss of claudin-5 from cardiomyocytes correlates with an abnormal “wavy” ultrastructural appearance of lateral membranes. Utrophin/dystrophin-deficient hearts do not show abnormalities in the levels or localization of the adherens junction proteins cadherin, alpha-catenin, or beta-catenin, nor in desmoplakin I and II or connexin-43¹⁴.

The claudin-5 gene lies within the critical region of the deletion on chromosome 22q11 that leads to the conotruncal cardiac abnormalities that characterize the contiguous gene syndrome known as velocardiofacial or DiGeorge syndrome^{18, 19}.

Therefore, there is a need to regulate the expression of claudin-5 protein for treatment of human diseases.

Considering the above-mentioned, there is also a need for therapeutic strategies to treat end-stage cardiomyopathy.

### SUMMARY OF THE INVENTION

The invention is based, at least in part, on the inventors' discovery that claudin-5 levels are statistically correlated with human end-stage cardiomyopathy.

Accordingly, in one aspect, the invention features methods of evaluating a subject, preferably a human, e.g., determining a subject's risk of developing end-stage cardiomyopathy. The methods include evaluating levels of claudin-5 in a subject. In one preferred embodiment, the cardiomyopathy is characterized by a specific alteration in claudin-5 and not by a general alteration of cell junction proteins.

In another aspect, there is provided a method for diagnosing end-stage cardiomyopathy that includes measuring expression of claudin-5 levels in a patient suspected of suffering from end-stage cardiomyopathy.

In another aspect, there is provided a method for inhibiting end-stage cardiomyopathy that includes administering an effective amount of a composition that effectively upregulates the expression of claudin-5 or inhibits the proteolysis of claudin-5. The method can be used as a therapeutic tool to prevent further end-stage cardiomyopathy.

In another aspect, there is provided a method for inhibiting end-stage cardiomyopathy that includes administering an effective amount of a composition comprising one or more claudin-5 upregulators.

In another aspect, there is provided a method for treating end-stage cardiomyopathy that includes administering to a patient in need thereof, a therapeutically effective amount of a pharmaceutical composition which comprises a substance that increases the activity of claudin-5, as an active ingredient.

In another aspect, there is provided a composition for affecting end-stage cardiomyopathy that comprises a claudin-5 upregulator. The pharmaceutical composition can include a pharmaceutically acceptable carrier. A method for preparing a pharmaceutical composition includes mixing a composition for affecting end-stage cardiomyopathy that comprises a claudin-5 upregulator and a pharmaceutically acceptable carrier.

A method for diagnosing a cardiac disorder in a patient in need thereof, comprises the steps of determining a level of expression of at least claudin-5 in cells of interest, and assessing whether claudin-5 is expressed at a level which is higher or lower than a predetermined level, where the cardiac disorder is implicated when claudin-5 is at or below the level which is lower than the predetermined level. In certain embodiments, the step of determining is carried out by exposing said cells of interest to at least one antibody recognizing claudin-5. In certain embodiments, the step of obtaining a sample of said cells of interest from said patient includes sampling cardiac tissue or peripheral blood.

In still another aspect, there is provided an animal model for examining end-stage cardiomyopathy that comprises administering one or more claudin-5 upregulators,

Various objects and advantages of this invention will become apparent to those skilled in the art from the following detailed description of the preferred embodiment, when read in light of the accompanying drawings.

### BRIEF DESCRIPTION OF THE DRAWINGS

FIG. 1 shows the western analysis using both polyclonal and monoclonal antibodies; on the same blots used to detect claudin-5 levels, an antibody raised against the cardiac-specific 40 kDa alpha-sarcomeric actin was used as a normalization control for equal loading and cardiomyocyte protein content.

FIG. 2 is a graph showing that the reduction in claudin-5 levels varied between samples, but fell into 2 major categories: 1) less than 25% of normal levels and, 2) between 25-75% of normal levels.

FIG. 3 is a graph showing the densitometric analysis of western blots confirming reductions of claudin-5 in ischemic and dilated cardiomyopathy explant samples.

### DETAILED DESCRIPTION OF THE PREFERRED EMBODIMENT

To confirm that cell junction protein alterations represent a critical step in the pathway from human cardiomyopathy to heart failure, the inventors herein determined the levels of the adherens junction proteins (cadherin, alpha-, beta-, and gamma-catenin; the desmosomal proteins: desmoplakin I and II; the gap junction protein connexin-43; and claudin-5) in end-stage failing cardiac explant samples from cardiomyopathy patients.

Reductions in the levels and phosphorylation of connexin-43 in a subset of cardiomyopathic samples were observed. In particular, the inventors herein now show that claudin-5, is dramatically reduced in 60% of human explant samples compared to non-failing controls. Importantly, reduced levels of claudin-5 can occur independently of connexin-43 alterations. Claudin-5 can also be reduced in samples with normal levels of dystrophin, known to be both a cause and result of cardiomyopathy. Notably, no other cell junction proteins showed an independent reduction in patient samples. While not wishing to be bound by theory, the inventors herein now believe that claudin-5 plays a critical role in the progression of cardiomyopathy to end stage heart failure in a large population of patients.

Methods

Patient Samples

Human heart samples used as controls were procured with the assistance of the Gift-of-Life Donor Program (Philadelphia, Pa.) in accordance with an approved research protocol. These samples were harvested after brain death from organ donors who had normal left ventricular ejection fractions, no history of heart failure and died from non-cardiac causes. In all cases, informed consent for the research use of donors' heart tissue was obtained in advance from donors' next-of-kin.

Samples of ventricular tissue were obtained from patients undergoing heart transplantation (61 patients) or implantation of a left ventricular assist device (one patient) according to an Ohio State University Institutional Review Board approved protocol. For patients undergoing heart transplantation, samples of myocardial tissue were obtained at the time of explant of the recipient's heart and tissue was immediately flash frozen in liquid nitrogen. In the patient undergoing implantation of a left ventricular assist device, a segment of myocardial tissue was excised from the left ventricular apex for cannula insertion and immediately flash frozen.

Of the 62 patients whose myocardial tissue was sampled, all had reduced ventricular ejection fractions ranging from 2% to 30% (mean+sd 16.8+7%). Thirty-six patients had ventricular dysfunction on the basis of ischemic heart disease and the remainder had non-ischemic dilated cardiomyopathy. Nineteen of the 62 patients were female; ten were self-identified as African American and the remainder as Caucasian.

Western Analysis

Explant samples were homogenized in Newcastle buffer (4M Urea, 75 mM Tris, pH 6.8, 3.8% SDS) and protein concentrations were determined using the Dc Protein Assay (Bio-Rad, Richmond, Calif.). Protein (25 or 50 g) was then run out on 6%, 8%, or 15% SDS-Polyacrylamide gel electrophoresis (SDS-PAGE) gels at 90V. Proteins were then transferred from SDS-PAGE gels to nitrocellulose (Schleicher and Schuell Bioscience, Keene, N.H.) at 80 V for 30, 70 or 150 minutes using a wet transfer apparatus (Bio-Rad, Richmond, Calif.). To determine protein levels of claudin-5, the inventors carried out western analysis using both polyclonal and monoclonal antibodies specific for this protein. For each sample, a mouse monoclonal antibody against sarcomeric actin, which has previously been used for the normalization of Cx43 in cardiac hypertrophy patient samples (Sigma A2172)²⁰ was used to control for equal loading and cardiomyocyte content. Detection of this 40 kDa actin control protein was determined on the same western blots used to detect the 23 kDa claudin-5 protein, and the larger catenin-proteins. After western transfer, blots were cut into two pieces just below the 32 kDa molecular weight marker. The blot containing the smaller molecular weight proteins was used to detect the 23 kDa claudin-5 protein, while the blot containing the higher molecular weight proteins was used to detect alpha-sarcomeric actin or connexin-43. Western blots were blocked in 5% nonfat milk in Tris-buffered saline plus 0.1% Tween-20 (TBST) and 1% normal goat serum (NGS). Blots were then incubated with affinity purified polyclonal or monoclonal primary antibodies diluted in TBST and 1% NGS for two hours at the following dilutions: rabbit anti-claudin-5 (Zymed, San Francisco, Calif.) 1:200; mouse anti-claudin-5 (Zymed) 1:200; rabbit anti-cadherin (Sigma, St. Louis, Mo.) 1:1000; rabbit anti-catenin (Sigma) 1:500; rabbit anti-catenin (Zymed) 1:1000; mouse anti-gamma-catenin (Zymed) 1:1000; rabbit anti-desmoplakin I and II (Serotec, Oxford, UK) 1:1000; rabbit anti-connexin-43 (Zymed) 1:500; mouse anti-actin (alpha-sarcomeric) (Sigma) 1:1000; mouse anti-dystrophin (Vector; C-terminal Dys-2; clone 6C5) 1:25; and mouse anti-dystrophin N-terminal (ManHinge 1B; clone 10F9). Blots were then washed 3×15 minutes in TBST and incubated with horse-radish peroxidase (HRP)-conjugated goat anti-rabbit or anti-mouse secondary antibody (Jackson Labs, West Grove, Pa.) for one hour at 1:10,000 in TBST plus 1% NGS. Enhanced chemiluminescence using the ECL plus kit (Amersham Pharmacia, Buckinghamshire, England) was used for detection of bound primary antibody. Westerns for claudin-5 (monoclonal and polyclonal), connexin-43, and alpha-sarcomeric actin controls were repeated at least three times for each sample.

Proteins from two non-failing samples were loaded on each gel to provide normative quantification specific for each gel. Protein levels of each failing sample were visually scored independently, by 2 investigators comparing each level with the two non-failing control samples and assigning scores as: equivalent to normal (3), moderately reduced (2), or severely reduced (1). The visual scoring of claudin-5 levels was tested in a subset of samples in which densitometric quantification of western blots normalized for actin was performed. Western blots were quantitated with Image Quant software for at least 5 representative samples from each group of claudin-5 reduced samples. After subtracting background pixels from an equal area measured for each protein band, total pixel intensity for claudin-5 bands were divided by total pixel intensity of actin for each sample. These normalized claudin-5 levels were expressed as a percentage of the average of normalized claudin-5 levels of the four non-failing control samples. Cut-offs were set at 25% of the mean of the non-failing controls for a score of 1, and at 75% of the mean of the non-failing controls for a score of 2. Since samples from non-failing hearts were separately loaded on each gel, they constituted a separate independent measurement environment. Therefore, the quantification of non-failing samples on each gel was pooled with samples from patients with cardiomyopathy that were visually scored as not differing from normal (3). Factorial analysis of variance was performed to test for significant differences in claudin-5 protein quantification in samples visually graded as normal (3), moderately reduced (2), and severely reduced (1). Due to limited control and failing sample material and the large sample size, accurate quantitation comparing the entire group of samples was prohibited based on the inability to run all 62 samples plus 4 controls on the same gel with dilutions of each sample required for quantification in the appropriate linear range. Precise scoring of each sample run in triplicate compared with two non-failing controls on the same gel was confirmed by densitometric quantification of a subset of samples as described above.

Results

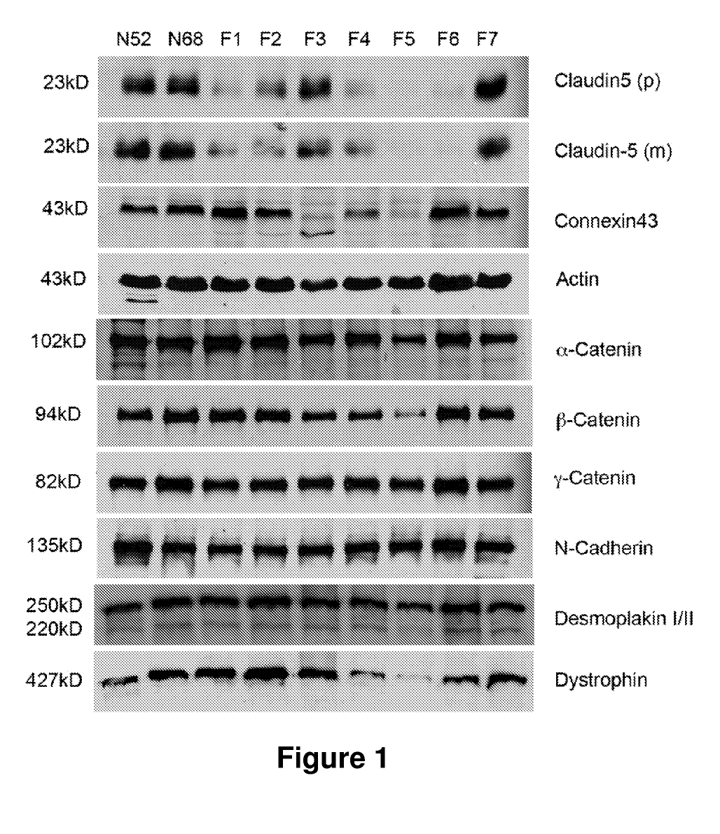

Claudin-5 levels are reduced in 60% of human cardiomyopathic samples. Sixty-two heart explant samples from patients diagnosed with either DCM or ICM were analyzed for levels of claudin-5 and other known cell junction proteins. Four non-failing heart samples, from hearts with normal ejection fractions were used as controls for all experiments. To determine protein levels of claudin-5, the inventors carried out western analysis using both polyclonal and monoclonal antibodies specific for this protein. On the same blots used to detect claudin-5 levels, the inventors used an antibody raised against the cardiac-specific 40 kDa alpha-sarcomeric actin as a normalization control for equal loading and cardiomyocyte protein content. All control and patient samples were found to have similar levels of alpha-sarcomeric actin per microgram of total protein content, indicating that the patient samples contained cardiomyocytes and not fibrotic tissue (FIG. 1). Results with monoclonal claudin-5 and polyclonal claudin-5 antibodies were consistent for each sample and identified 37 samples with reductions of claudin-5 compared to the levels present in the four control samples (FIG. 1).

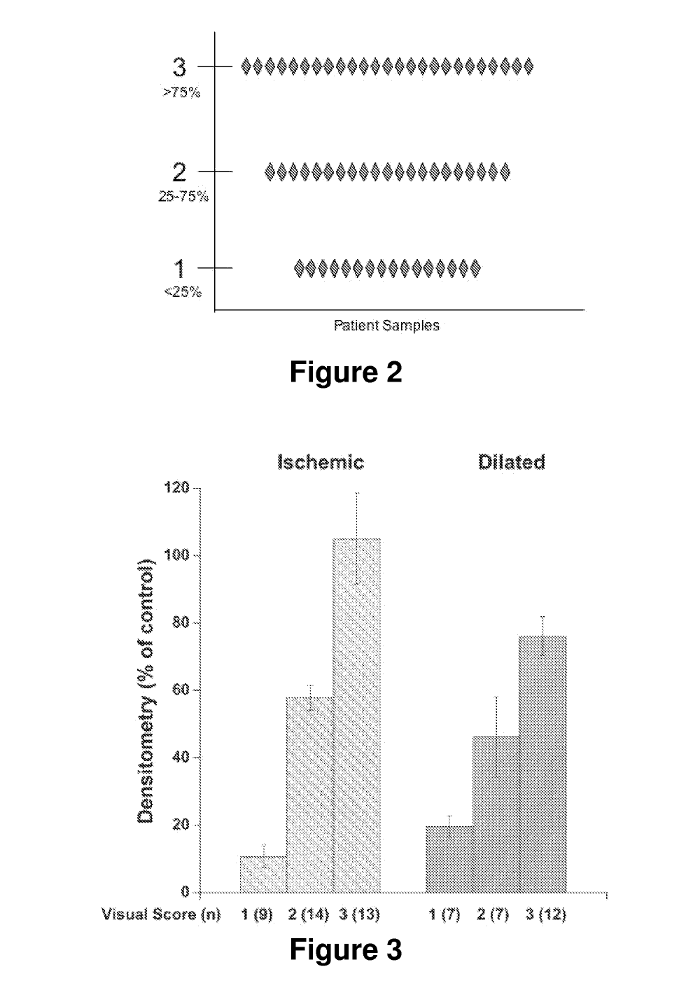

This number of samples represents 60% of the patient samples, showing a high incidence of claudin-5 alterations in the cardiomyopathic patient population. The reduction in claudin-5 levels varied between samples, but fell into 2 major categories: 1) less than 25% of normal levels and, 2) between 25-75% of normal levels, as shown in the graph in FIG. 2.

Samples with more than 75% of claudin-5 levels present in the average of the four normal controls, or equal to 90% of the lowest normal control, were considered in the normal range as to not overestimate the affected population. Factorial ANOVA showed a significant (p<0.02) difference in quantification of claudin-5 in samples visually graded as normal (3), moderately reduced (2), and severely reduced (1).

Decreased claudin-5 levels were present in samples from patients of different sexes, races, and with different etiologies including DCM versus ICM, and diabetic versus non-diabetic. This association with a diverse patient population emphasizes that claudin-5 may represent a common pathway to heart failure.

Reduced Levels of Claudin-5 can Occur Independently of Connexin-43 Alterations.

The cell junction protein connexin-43 is the major component of gap junctions and is useful to show altered levels or phosphorylation associated with a variety of cardiac dysfunction, including end-stage cardiomyopathy. To confirm that the patient population contained reduced connexin-43 levels or phosphorylation, the inventors compared connexin-43 levels in explant samples with controls. Western analysis shows that a subset of failing cardiac samples contains reduced levels of connexin-43 (FIG. 1: F3, F4, F5).

Connexin-43 alterations were present both independent from (FIG. 1: F3), and in combination with (FIG. 1: F4, F5) reduced claudin-5 levels. Reductions in claudin-5 could occur without connexin-43 alterations (FIG. 1: F1, F2), showing that claudin-5 decreases are independent of connexin-43 changes.

Reduced Claudin-5 Levels are not Associated with a General Reduction in Cell Junction Proteins.

To distinguish whether the observed decrease in claudin-5 in cardiomyopathic patient heart samples is specific, or is associated with a more general alteration of cell junction proteins, the inventors investigated other cell junction protein levels in the same patient samples. The inventors determined the levels of cell junction proteins: N-cadherin, alpha-, beta-, and gamma-catenin that compose adherens junctions, and Desmoplakin I and II that compose desmosomes. Alpha-catenin and beta-catenin were never present at reduced levels independent of claudin-5 or connexin-43 reductions. However, these two catenins were reduced in a small percentage (˜12-15%) of total failing samples, all of which also had reduced levels of claudin-5 or connexin-43. Cadherin, gamma-catenin, and desmoplakin were present at levels equivalent or similar to controls in 60, 58, and 59 of the 62 patient samples, respectively. In one explant sample (78), all cell junction proteins, except desmoplakin, were reduced in combination with reduced levels of claudin-5 and connexin-43 (FIG. 1, lane F5).

In a second explant sample with reduced levels of several cell junction proteins (40) including connexin-43, claudin-5 levels were equivalent to those in non-failing controls.

Claudin-5 Levels are not Dependent on Dystrophin.

The inventors identified reduced claudin-5 levels in utrophin/dystrophin-deficient mice (noting that dystrophin has been shown to be decreased in patients with DCM²¹). To determine whether claudin-5 alterations were linked with dystrophin alterations, the inventors investigated the levels of dystrophin protein in patient samples with decreased levels of claudin-5. Reductions of claudin-5 can be present together with reductions of dystrophin (FIG. 1: F4, F5, F6); however, claudin-5 decreases can be independent of decreases in dystrophin (FIG. 1: F1, F2, F6).

These data confirm that claudin-5 decreases in human explant samples is independent of all other cell junction proteins and of previously reported decreases in dystrophin.

Discussion

The inventors now show that specific decreases of claudin-5 are associated with at least 60 percent of end-stage heart failure. Decreased claudin-5 levels can occur independently from other previously identified protein level changes associated with cardiomyopathy including alterations of connexin-43 and dystrophin. Claudin-5 decreases were present in end-stage cardiac explant samples from patients of different sexes and races, suggesting that this change is not genetically linked to a specific population or sex.

Explant samples from patients diagnosed with both ICM and DCM and from those patients with and without a history of diabetes show decreased levels of claudin-5. While not wishing to be bound by theory, the inventors herein now believe that claudin-5 is part of a pathway leading from cardiomyopathy of different etiologies to the final common endpoint of heart failure. The patient sample population mirrored previous observations of altered connexin-43 levels and phosphorylation, confirming that the population was representative of other previously published observations.

However, claudin-5 levels showed greater decreases in more patient samples as connexin-43, showing that claudin-5 is now believed by the inventors herein to represent a more common mechanism associated with heart failure. It is of particular interest that only one sample contained reduced levels of all adherens junction proteins, but no claudin-5 decrease, underscoring that alterations of claudin-5 at cardiomyocyte lateral membranes represent a much more common feature of end-stage failing explant cardiac samples than intercalated disc alterations.

The inventors' observations of dystrophin decreases are consistent with the observations that primary mutations in dystrophin cause Duchenne muscular dystrophy and at least 95% of patients have DCM. Specific mutations affecting dystrophin expression in heart lead primarily to DCM and dystrophin has been shown to undergo proteolysis in samples from some heart failure patients. The inventors herein show that claudin-5 decreases can occur independently of dystrophin decreases, supporting that claudin-5 is involved in a pathway independent of dystrophin. In contrast to a previous report²¹, where dystrophin levels detected by only an N-terminal but not a mid-rod or a C-terminal antibody were affected, the data herein show similar decreases of dystrophin levels detected with a C-terminal antibody (FIG. 1).

The inventors herein confirmed that samples with reduced levels of dystrophin detected with the C-terminal antibody were also detected with an N-terminal dystrophin antibody.

Claudin-5 was identified as a candidate for involvement in the heart failure phenotype of utrophin/dystrophin-deficient mice due to its decreased gene expression detected in a microarray experiment. Claudin-5 was the only member of the claudin protein family that showed altered gene expression in this global analysis. While the inventors herein realize that this data does not rule out the possibility of other claudins in the much broader category of human heart failure, the inventors herein have now identified claudin-5 as a pathway for the development for treatment of cardiac diseases.

The detection of expression of claudin-5 in cardiomyocytes and their involvement in heart failure provides a useful mechanism to diagnose end-stage heart failure.

As shown in FIG. 3, the densitometric analysis of western blots confirms reductions of claudin-5 in ischemic and dilated cardiomyopathy explant samples. Quantification by densitometry of a subset of the 62 human cardiac samples confirmed the scoring analysis of claudin-5 levels. (n) refers to the number of total samples scored as 1, 2, or 3 separated by ischemic or dilated cardiomyopathy diagnosis. Densitometry was performed on a subset of samples from each category where at least 2 non-failing controls run on the same gel could be used as a reference. Claudin-5 values normalized for actin for each sample were divided by the average of nonnalized claudin-5 values from non-failing controls. In both ischemic and dilated cardiomyopathy samples, a score of 1 reflects greatly reduced claudin-5 level, usually between 10 and 20% claudin-5 levels present in controls. Samples visually scored as 2 contain levels of claudin-5 approximately half that of non-failing controls, and those scored as 3 were equal or slightly less than controls. Thus, conservatively scored, 60% of the samples showed moderate or severe reductions in claudin-5. The number of samples used for densitometry (phenotype: score 1, 2, and 3) were for ICM: 6, 8, and 5; and for DCM: 4, 4, and 6. T-test analysis revealed no significant difference between the ICM and DCM densitometry analysis (ICM vs. DCM, score 1 p=0.14, score 2 p=0.29, score 3 p=0.20), whereas within each group all differences between the scores (1 vs. 2 vs. 3) were significant (p<0.05).

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof. Therefore, it is intended that the invention not be limited to the particular embodiment disclosed herein contemplated for carrying out this invention, but that the invention will include all embodiments falling within the scope of the claims.

### REFERENCES

1. Towbin J A, Bowles N E. The failing heart. Nature. 2002; 415:227-33.
2. Ehler E, Horowits R, Zuppinger C, Price R L, Perriard E, Leu M, Caroni P, Sussman M, Eppenberger H M, Perriard J C. Alterations at the intercalated disk associated with the absence of muscle LIM protein. J Cell Biol. 2001; 153:763-72.
3. Ferreira-Cornwell M C, Luo Y, Narula N, Lenox J M, Lieberman M, Radice G L. Remodeling the intercalated disc leads to cardiomyopathy in mice misexpressing cadherins in the heart. J Cell Sci. 2002; 115:1623-34.
4. Li J, Patel V V, Kostetskii I, Xiong Y, Chu A F, Jacobson J T, Yu C, Morley G E, Molkentin J D, Radice G L. Cardiac-specific loss of N-cadherin leads to alteration in connexins with conduction slowing and arrhythmogenesis. Circ Res. 2005; 97:474-81.
5. Perriard J C, Hirschy A, Ehler E. Dilated cardiomyopathy: a disease of the intercalated disc? Trends Cardiovasc Med. 2003; 13:30-8.
6. Li J, Patel V V, Radice G L. Dysregulation of cell adhesion proteins and cardiac arrhythmogenesis. Clin Med Res. 2006; 4:42-52.
7. Maeda M, Holder E, Lowes B, Valent S, Bies R D. Dilated cardiomyopathy associated with deficiency of the cytoskeletal protein metavinculin. Circulation. 1997; 95:17-20.
8. Schaper J, Froede R, Hein S, Buck A, Hashizume H, Speiser B, Friedl A, Bleese N. Impairment of the myocardial ultrastructure and changes of the cytoskeleton in dilated cardiomyopathy. Circulation. 1991; 83:504-14.
9. Dupont E, Matsushita T, Kaba R A, Vozzi C, Coppen S R, Khan N, Kaprielian R, Yacoub M H, Severs N J. Altered connexin expression in human congestive heart failure. J Mol Cell Cardiol. 2001; 33:359-71.
10. Peters N S, Green C R, Poole-Wilson P A, Severs N J. Reduced content of connexin43 gap junctions in ventricular myocardium from hypertrophied and ischemic human hearts. Circulation. 1993; 88:864-75.
11. Kostin S, Rieger M, Dammer S, Hein S, Richter M, Klovekorn W P, Bauer E P, Schaper J. Gap junction remodeling and altered connexin43 expression in the failing human heart. Mol Cell Biochem. 2003; 242:135-44.
12. Protonotarios N I, Tsatsopoulou A A, Gatzoulis K A. Arrhythmogenic right ventricular cardiomyopathy caused by a deletion in plakoglobin (Naxos disease). Card Electrophysiol Rev. 2002; 6:72-80.
13. McGrath J A. Inherited disorders of desmosomes. Australas J Dermatol. 2005; 46:221-9.
14. Sanford J L, Edwards J D, Mays T A, Gong B, Merriam A P, Rafael-Fortney J A. Claudin-5 localizes to the lateral membranes of cardiomyocytes and is altered in utrophin/dystrophin-deficient cardiomyopathic mice. J Mol Cell Cardiol. 2005; 38:323-32.
15. Hainsey T A, Senapati S, Kuhn D E, Rafael J A. Cardiomyopathic features associated with muscular dystrophy are independent of dystrophin absence in cardiovasculature. Neuromuscul Disord. 2003; 13:294-302.
16. Deconinck A E, Rafael J A, Skinner J A, Brown S C, Potter A C, Metzinger L, Watt D J, Dickson J G, Tinsley J M, Davies K E. Utrophin-dystrophin-deficient mice as a model for Duchenne muscular dystrophy. Cell. 1997; 90:717-27.
17. Janssen P M, Hiranandani N, Mays T A, Rafael-Fortney J A. Utrophin deficiency worsens cardiac contractile dysfunction present in dystrophin-deficient mdx mice. Am J Physiol Heart Circ Physiol. 2005; 289:H2373-8.
18. Lindsay E A. Chromosomal microdeletions: dissecting del22q11 syndrome. Nat Rev Genet. 2001; 2:858-68.
19. Sirotkin H, Morrow B, Saint-Jore B, Puech A, Das Gupta R, Patanjali S R, Skoultchi A, Weissman S M, Kucherlapati R. Identification, characterization, and precise mapping of a human gene encoding a novel membrane-spanning protein from the 22q11 region deleted in velo-cardio-facial syndrome. Genomics. 1997; 42:245-51.
20. Kostin S, Dammer S, Hein S, Klovekorn W P, Bauer E P, Schaper J. Connexin 43 expression and distribution in compensated and decompensated cardiac hypertrophy in patients with aortic stenosis. Cardiovasc Res. 2004; 62:426-36.
21. Vatta M, Stetson S J, Perez-Verdia A, Entman M L, Noon G P, Torre-Amione G, Bowles N E, Towbin J A. Molecular remodelling of dystrophin in patients with end-stage cardiomyopathies and reversal in patients on assistance-device therapy. Lancet. 2002; 359:936-41.
22. Miyamori H, Takino T, Kobayashi Y, Tokai H, Itoh Y, Seiki M, Sato H. Claudin promotes activation of pro-matrix metalloproteinase-2 mediated by membrane-type matrix metalloproteinases. J Biol Chem. 2001; 276:28204-11.
23. Yang Y, Estrada E Y, Thompson J F, Liu W, Rosenberg G A. Matrix metalloproteinase-mediated disruption of tight junction proteins in cerebral vessels is reversed by synthetic matrix metalloproteinase inhibitor in focal ischemia in rat. J Cereb Blood Flow Metab. 2007; 27:697-709.
24. Oku N, Sasabe E, Ueta E, Yamamoto T, Osaki T. Tight junction protein claudin-1 enhances the invasive activity of oral squamous cell carcinoma cells by promoting cleavage of laminin-5 gamma2 chain via matrix metalloproteinase (MMP)-2 and membrane-type MMP-1. Cancer Res. 2006; 66:5251-7.
25. Schwartzkopff B, Fassbach M, Pelzer B, Brehm M, Strauer B E. Elevated serum markers of collagen degradation in patients with mild to moderate dilated cardiomyopathy. Eur J Heart Fail. 2002; 4:439-4.
26. Thomas C V, Coker M L, Zellner J L, Handy J R, Crumbley A J, 3rd, Spinale F G. Increased matrix metalloproteinase activity and selective upregulation in LV myocardium from patients with end-stage dilated cardiomyopathy. Circulation. 1998; 97:1708-15.
27. Tiyyagura S R, Pinney S P. Left ventricular remodeling after myocardial infarction: past, present, and future. Mt Sinai J Med. 2006; 73:840-51.
28. Gallagher G L, Jackson C J, Hunyor S N. Myocardial extracellular matrix remodeling in ischemic heart failure. Front Biosci. 2007; 12:1410-9.
29. Honda M, Nakagawa S, Hayashi K, Kitagawa N, Tsutsumi K, Nagata I, Niwa M. Adrenomedullin improves the blood-brain barrier function through the expression of claudin-5. Cell Mol Neurobiol. 2006; 26:109-18.
30. Ishizaki T, Chiba H, Kojima T, Fujibe M, Soma T, Miyajima H, Nagasawa K, Wada I, Sawada N. Cyclic AMP induces phosphorylation of claudin-5 immunoprecipitates and expression of claudin-5 gene in blood-brain-barrier endothelial cells via protein kinase A-dependent and -independent pathways. Exp Cell Res. 2003; 290:275-88.
31. Martin T A, Das T, Mansel R E, Jiang W G. Synergistic regulation of endothelial tight junctions by antioxidant (Se) and polyunsaturated lipid (GLA) via Claudin-5 modulation. J Cell Biochem. 2006; 98:1308-19.
32. Dimitrijevic O B, Stamatovic S M, Keep R F, Andjelkovic A V. Effects of the chemokine CCL2 on blood-brain barrier permeability during ischemia-reperfusion injury. J Cereb Blood Flow Metab. 2006; 26:797-810.

1. A method for diagnosing end-stage cardiomyopathy comprising measuring expression of claudin-5 levels in a patient suspected of suffering from end-stage cardiomyopathy, and comparing the claudin-5 levels to a control level, wherein an increase in the level of claudin-5 relative to the control level is indicative of the patient having end-stage cardiomyopathy.

2. A method for inhibiting end-stage cardiomyopathy comprising administering an effective amount of a composition that effectively upregulates the expression of claudin-5 or inhibits degradation of claudin-5.

3. The method of claim 2 useful as a therapeutic tool to prevent further end-stage cardiomyopathy.

4. A method for inhibiting end-stage cardiomyopathy comprising administering an effective amount of a composition comprising one or more claudin-5 upregulators.

5. A method for treating end-stage cardiomyopathy comprising administering to a patient in need thereof, a therapeutically effective amount of a pharmaceutical composition which comprises a substance that upregulates claudin-5, as an active ingredient.

6. A composition for affecting end-stage cardiomyopathy comprising a claudin-5 upregulator or an inhibitor of its degradation.

7. A pharmaceutical composition comprising the composition of claim 6 and a pharmaceutically acceptable carrier.

8. A method for preparing a pharmaceutical composition comprising mixing the composition of claim 6 and a pharmaceutically acceptable carrier.

9. An animal model for examining end-stage cardiomyopathy comprising administering one or more claudin-5 upregulators to an animal experiencing end-stage cardiomyopathy.

10. A method for diagnosing a cardiac disorder in a patient in need thereof, comprising the steps of determining a level of expression of at least claudin-5 in cells of interest, and assessing whether claudin-5 is expressed at a level which is higher or lower than a predetermined level, where the cardiac disorder is implicated when claudin-5 is at or below the level which is lower than the predetermined level.

11. The method of claim 10, wherein the step of determining is carried out by exposing said cells of interest to at least one antibody recognizing claudin-5.

12. The method of claim 10, further comprising the step of obtaining a sample of the cells of interest from the patient.

13. The method of claim claim 1, further including comparing one or more cell junction proteins selected fro: adheren junction proteins (cadherin, alpha-, beta-, and gamma-catenin); desmosomal proteins: desmoplakin I and II; and gap junction protein connexin-43) to a control level, wherein the cell junction protein are not elevated as compared to the control levels.

14. The method of claim claim 13, further including comparing connexin-43 cell junction protein to a control level, wherein the connexin-43 cell junction protein is not elevated as compared to the control level.
